(19) United States
(12) Patent Application Publication
(10) Pub. No.: US 2016/0310126 A1
(43) Pub. Date: Oct. 27, 2016

(54) APPARATUS AND METHODS FOR REATTACHING DETACHED TISSUE TO HARD TISSUE

(71) Applicant: CHIA-WEI LIN, Tainan City (TW)

(72) Inventors: CHIA-WEI LIN, Tainan City (TW); CHI-JU LEE, Kaohsiung City (TW); YI-HUNG LIN, Kaohsiung City (TW); LING-YI HUANG, Taipei City (TW)

(21) Appl. No.: 15/133,002

(22) Filed: Apr. 19, 2016

Related U.S. Application Data
(60) Provisional application No. 62/150,526, filed on Apr. 21, 2015.

(51) Int. Cl. A61B 17/04

(52) U.S. Cl. CPC A61B 17/0401; A61B 2017/0404; A61B 2017/0464; A61B 2017/044; A61B 2017/0427

(57) ABSTRACT

An apparatus for reattaching a detached tissue to a hard tissue includes a suture anchor, at least one button, at least one first suture, a fastener, and at least one second suture. The suture anchor is fixed on a hard tissue. The at least one button is configured to press a detached tissue on the hard tissue. The at least one first suture is secured to the suture anchor and penetrates the detached tissue and a first hollow portion of the button. The fastener is fixed on the hard tissue. The at least one second suture is secured to the fastener and penetrates a second hollow portion of the button, wherein the second suture and the first suture are knotted on the bisecting rod of the button, thereby generating a pressure on the button to uniformly press the detached tissue on the hard tissue.

### CROSS-REFERENCE TO RELATED APPLICATIONS

This application claims the benefit of U.S. Provisional Application No. 62/150,526, filed Apr. 21, 2015, the entire disclosure of which is incorporated by reference herein.

### FIELD

The disclosure relates to an apparatus and a method for reattaching a detached tissue to a hard tissue.

### BACKGROUND

The rotator cuff is a group of muscles at an innermost layer of the shoulder joint, including supraspinatus, infraspinatus, teres minor and subscapularis, is a core muscle group of the shoulder joint, and has a main function of stabilizing humerus when the shoulder joint moves. When suffering from a trauma or being repetitively used, for example, suffering from collision or continuously lifting an arm to clean a high place, the rotator cuff may be worn, or even suffer from fibrosis and tear. Moreover, aging is also one important pathogenic factor. According to statistics, approximately 13% of the population at an age greater than 50 suffers from rotator cuff tendon tear, and over half of the population at an age greater than 80 is diagnosed with a rotator cuff tendon injury. After the rotator cuff injury, possible clinical manifestations include: being painful (sleep may be affected), being incapable of completing an action over the shoulder, being difficult in getting dressed and other disorders of repetitive shoulder function movements.

The extent of the rotator cuff tendon injury or tear may be divided into three stages, and symptoms and therapies of the rotator cuff tendon injury or tear are shown in Table 1.

TABLE 1

| Grade | Symptom | Therapy |
|---|---|---|
| I | edema nearby the muscle tendon, | physical therapy and drug |
| | and bleeding (usually at an age | control |
| | less than 25) | |
| II | tendinitis/bursitis and fibrosis | physical therapy and drug |
| | (usually at an age of 25 to 40) | control, or taking surgery |
| | | intervention into consid- |
| | | eration in case of severity |
| III | bone spur and muscle tendon tear | putting stress on surgery |
| | (usually at an age greater than 40) | intervention |

In Table 1, the part of the surgery intervention therapies is further roughly classified into a conventional open type, a mini open type and an arthroscopic method, as shown in Table 2.

TABLE 2

| | Conventional | Mini | |
|---|---|---|---|
| | open type | open type | Arthroscopy |
| shoulder joint | X | ◯ | ◯ |
| assessment | | | |
| wound size | 4 to 6 cm | 3 to 4 cm | 0.4 to |
| | | | 0.7 cm |
| deltoid | excision | pushing aside | tiny |
| invasion extent | and repair | | |
| repair manner | bone | bone tunnel | single-row or |
| | tunnel | fixation | double-row suture |
| | fixation | or single-row | anchor, and bone |
| | | suture anchor | tunnel fixation |
| postoperative | ◯ | ◯ | ◯ |
| initial passive | | | |
| movement | | | |
| postoperative | X | ◯ | ◯ |
| initial active | | | |
| assisted movement | | | |
| postoperative | X | ◯ | ◯ |
| initial active | | | |
| movement | | | |
| postoperative | moderate | moderate | mild |
| pain | to severe | | |
| rehabilitation | 9 to 12 | 4 to 12 | 3 to 6 |
| period | months | months | months |

Because the wound is small, and the postoperative recovery is quick, more orthopedists tend to perform suture anchor by using the arthroscopy to assist in fixing the muscle tendon to a bone, and this surgical manner is slowly used as a standard process of rotator cuff repair. However, the suture anchor is still incapable of completely fixing the rotator cuff, and a case of failure still occurs, for example, a fixing screw is pulled out due to osteoporosis, or the rotator cuff tendon is torn again because of a suture.

### SUMMARY OF THE INVENTION

In accordance with one aspect of the present disclosure, an apparatus for reattaching a detached tissue to a hard tissue includes a suture anchor, at least one button, at least one first suture, a fastener, and at least one second suture. The suture anchor is fixed on a hard tissue. The at least one button is configured to press a detached tissue on the hard tissue, wherein the button includes a hollow portion and a bisecting rod disposed within the hollow portion for dividing the hollow portion into a first hollow portion and a second hollow portion. The at least one first suture is secured to the suture anchor and penetrates the detached tissue and the first hollow portion of the button. The fastener is fixed on the hard tissue. The at least one second suture is secured to the fastener and penetrates the second hollow portion of the button, wherein the second suture and the first suture are knotted on the bisecting rod of the button, thereby generating a pressure on the button to uniformly press the detached tissue on the hard tissue.

In accordance with another aspect of the present disclosure, a method for reattaching a detached tissue to a hard tissue includes operation in which a suture anchor is fixed on a hard tissue and at least one first suture is secured to the suture anchor and penetrates a detached tissue. The method continues with operation in which a fastener is fixed on the hard tissue and at least one second suture is secured to the fastener. The method continues with operation in which at least one button is configured to press the detached tissue on the hard tissue, wherein the button includes a hollow portion and a bisecting rod disposed within the hollow portion for dividing the hollow portion into a first hollow portion and a second hollow portion. The method continues with operation in which the first suture penetrates the first hollow portion of the button and the second suture penetrates the second hollow portion of the button. The method continues with operation in which the second suture and the first suture are knotted on the bisecting rod of the button, thereby generating a pressure on the button to uniformly press the detached tissue on the hard tissue.

### BRIEF DESCRIPTION OF THE DRAWINGS

Aspects of the present disclosure are understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 is a schematic view of an apparatus in accordance with some embodiments of the present disclosure.

FIG. 2 illustrates a schematic view of an apparatus for reattaching a detached tissue to a hard tissue in accordance with some embodiments of the present disclosure.

FIG. 3 is a side view of a button contacted with a detached tissue in accordance with some embodiments of the present disclosure.

FIG. 4 is a schematic view of an apparatus in accordance with some embodiments of the present disclosure.

FIG. 5 is a flow diagram illustrating a method for reattaching a detached tissue to a hard tissue in accordance with some embodiments of the present disclosure.

FIGS. 6A to 6E illustrate behavioral views of an apparatus in various stages corresponding to the method of FIG. 5.

### DETAILED DESCRIPTION OF THE INVENTION

It is to be understood that the following disclosure provides many different embodiments or examples, for implementing different features of various embodiments. Specific examples of components and arrangements are described below to simplify the present disclosure. The present disclosure may, however, be embodied in many different forms and should not be construed as being limited to the embodiments set forth herein; rather, these embodiments are provided so that this description will be thorough and complete, and will fully convey the present disclosure to those of ordinary skill in the art. It will be apparent, however, that one or more embodiments may be practiced without these specific details.

In addition, the present disclosure may repeat reference numerals and/or letters in the various examples. This repetition is for the purpose of simplicity and clarity and does not in itself dictate a relationship between the various embodiments and/or configurations discussed.

It will be understood that singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms; such as those defined in commonly used dictionaries, should be interpreted as having a meaning that is consistent with their meaning in the context of the relevant art and the present disclosure, and will not be interpreted in an idealized or overly formal sense unless expressly so defined herein.

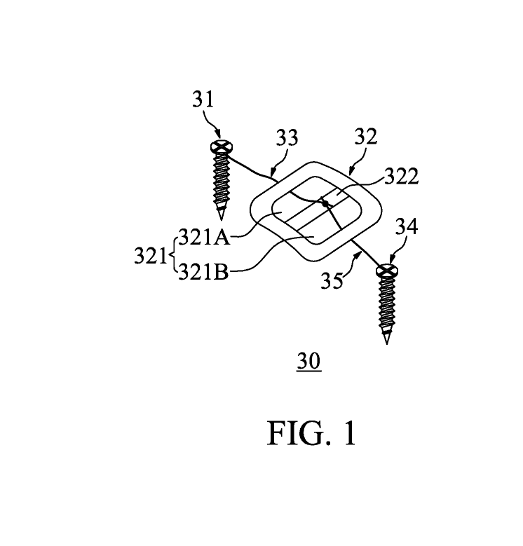

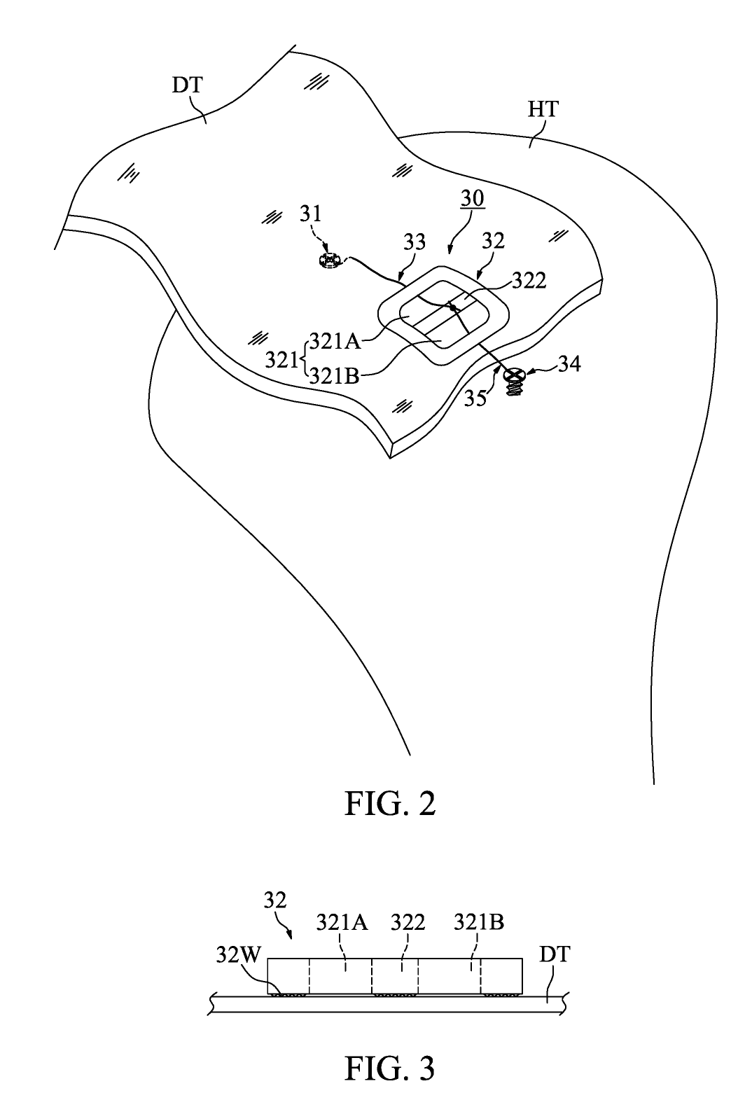

Referring to FIGS. 1 and 2, an apparatus 30 is designed to reattach a detached tissue DT on a hard tissue HT. In some embodiments, the detached tissue DT is a soft tissue such as rotator cuff, and the hard tissue HT is humerus. In some embodiments, the detached tissue DT can also be a hard tissue such as bone chip.

The apparatus 30 includes a suture anchor 31 fixed on the hard tissue HT.

At least one button 32 is configured to press the detached tissue DT on the hard tissue HT. The button 32 includes a hollow portion 321 and a bisecting rod 322 disposed within the hollow portion 321 for dividing the hollow portion 321 into a first hollow portion 321A and a second hollow portion 321B.

Referring to FIG. 3, in order to improve the contact force between the button 32 and the detached tissue DT, the button 32 can include a sawtooth structure 32W contacted with the detached tissue DT.

Referring to FIGS. 1 and 2 again, at least one first suture 33 is secured to the suture anchor 31 and penetrates the detached tissue DT and the first hollow portion 321A of the button 32.

A fastener 34 is fixed on the hard tissue HT. In some embodiments, the fastener 34 can be a suture anchor or a pushlock.

At least one second suture 35 is secured to the fastener 34 and penetrates the second hollow portion 321B of the button 32. The second suture 35 and the first suture 33 are knotted on the bisecting rod 322 of the button 32, thereby generating a pressure on the button 32 to uniformly press the detached tissue DT on the hard tissue HT.

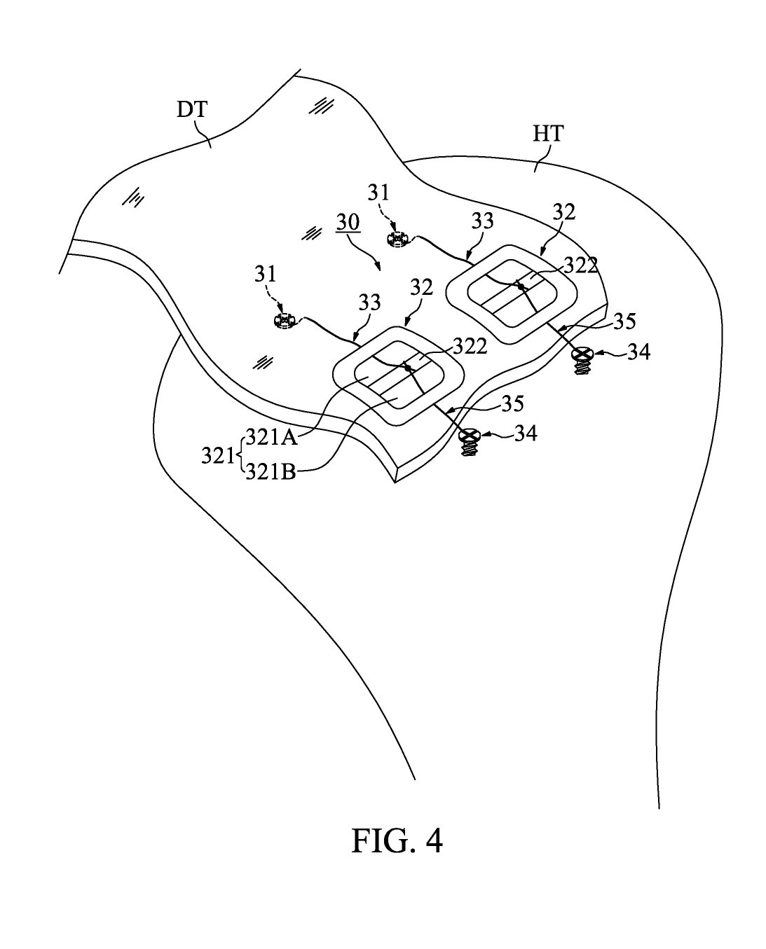

In the embodiment of FIG. 4, structural features of the apparatus 30 are basically the same as those of the embodiment of FIG. 2, and a difference therebetween only lies in that two suture anchors 31 are fixed on the hard tissue HT. Two buttons 32 are configured to press the detached tissue DT on the hard tissue HT. Two first sutures 33 are respectively secured to each suture anchor 31 and penetrate the first hollow portion 321A of each button 32. Two fasteners 34 are fixed on the hard tissue HT. Two second sutures 35 are respectively secured to each fastener 34 and penetrate the second hollow portion 321B of each button 32.

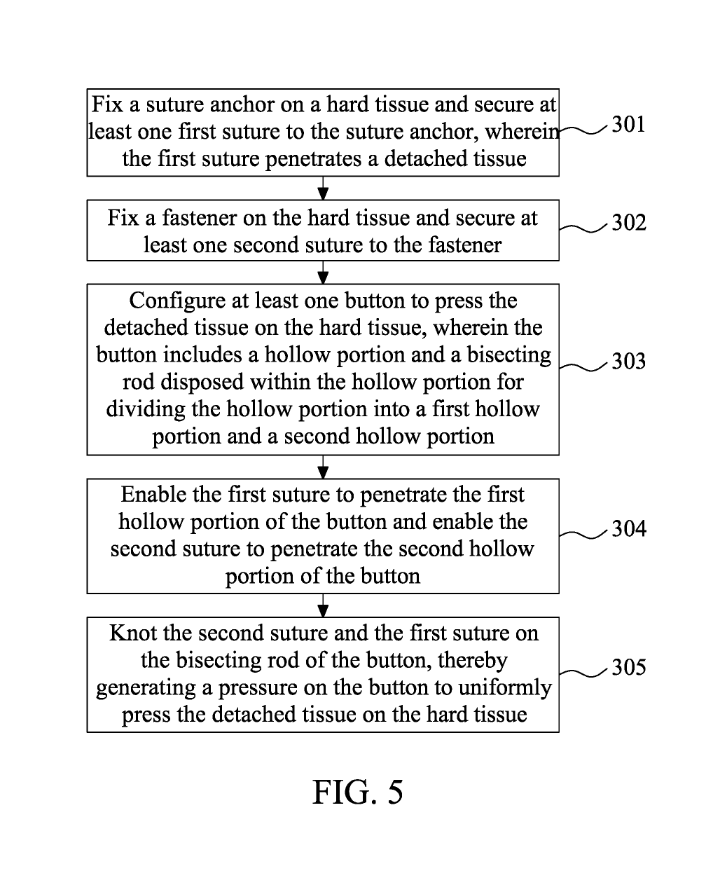

Referring to FIG. 5, a method includes operation 301 in which a suture anchor is fixed on a hard tissue and at least one first suture is secured to the suture anchor and penetrates a detached tissue. The method continues with operation 302 in which a fastener is fixed on the hard tissue and at least one second suture is secured to the fastener. The method continues with operation 303 in which at least one button is configured to press the detached tissue on the hard tissue, wherein the button includes a hollow portion and a bisecting rod disposed within the hollow portion for dividing the hollow portion into a first hollow portion and a second hollow portion. The method continues with operation 304 in which the first suture penetrates the first hollow portion of the button and the second suture penetrates the second hollow portion of the button. The method continues with operation 305 in which the second suture and the first suture are knotted on the bisecting rod of the button, thereby generating a pressure on the button to uniformly press the detached tissue on the hard tissue. The various operations of FIG. 5 are discussed below in more detail in association with behavioral views corresponding to the operations of the flow diagram. Although the operation sequence shown in FIG. 5 is 301, 302, 303, 304 and 305, various operation sequences are within the contemplated scope of the present disclosure.

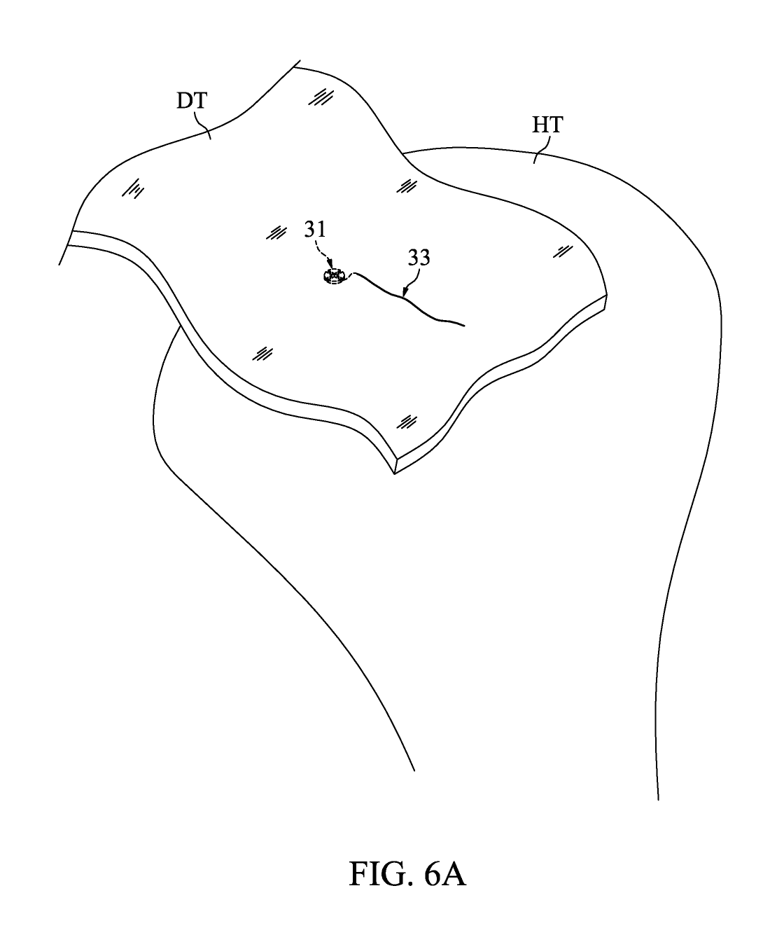

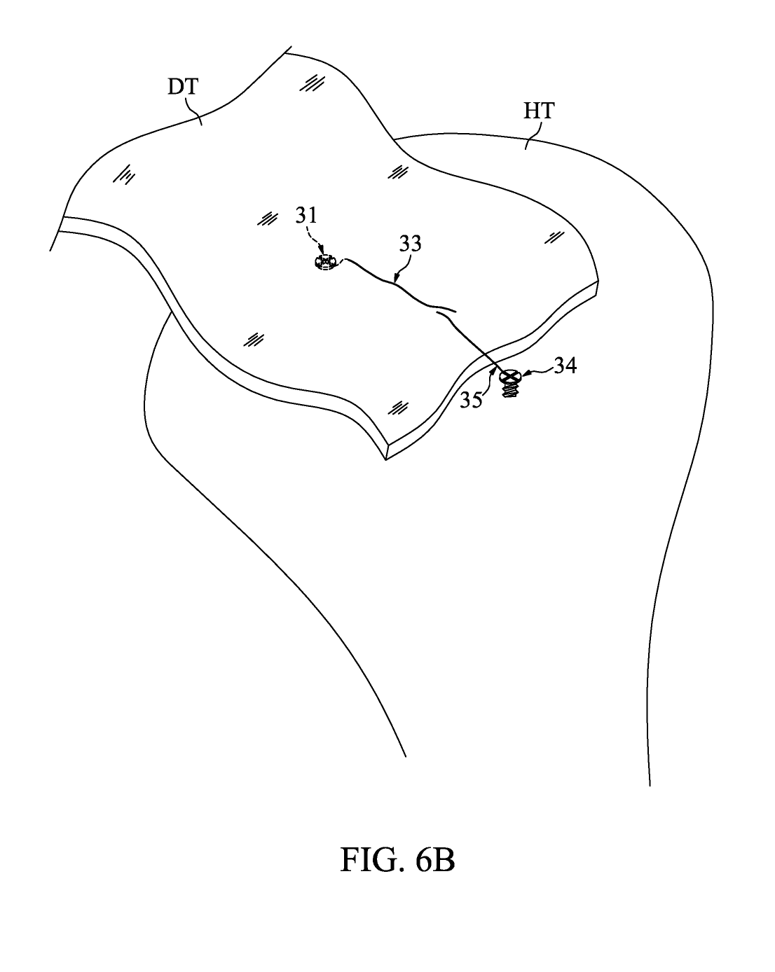

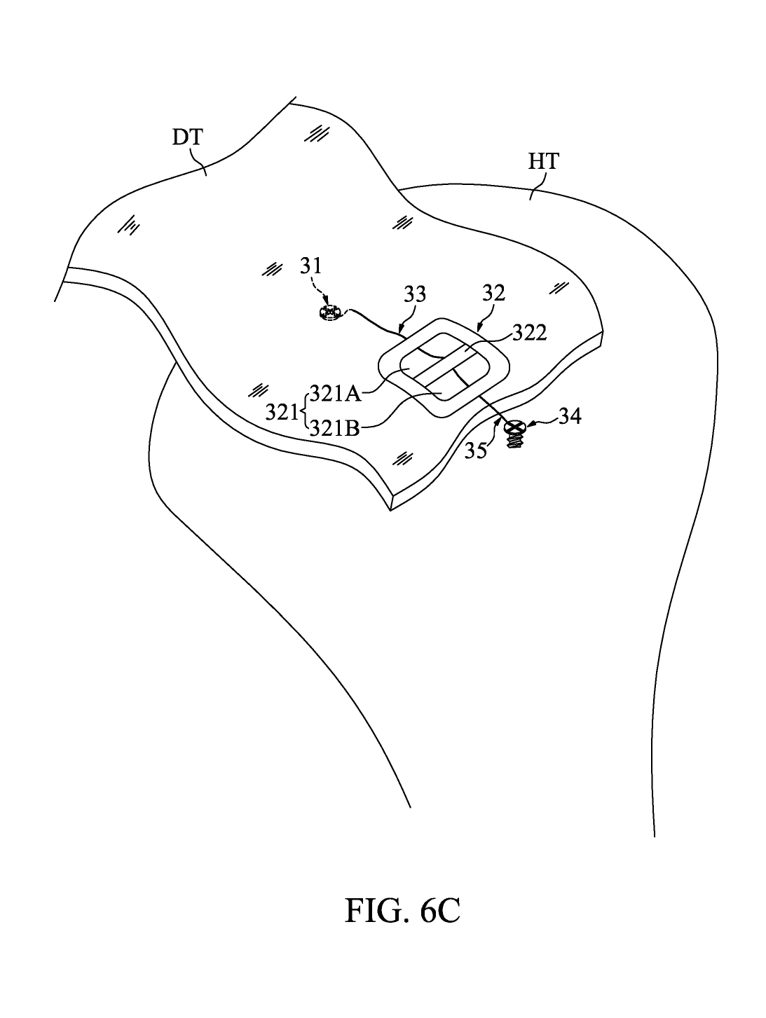

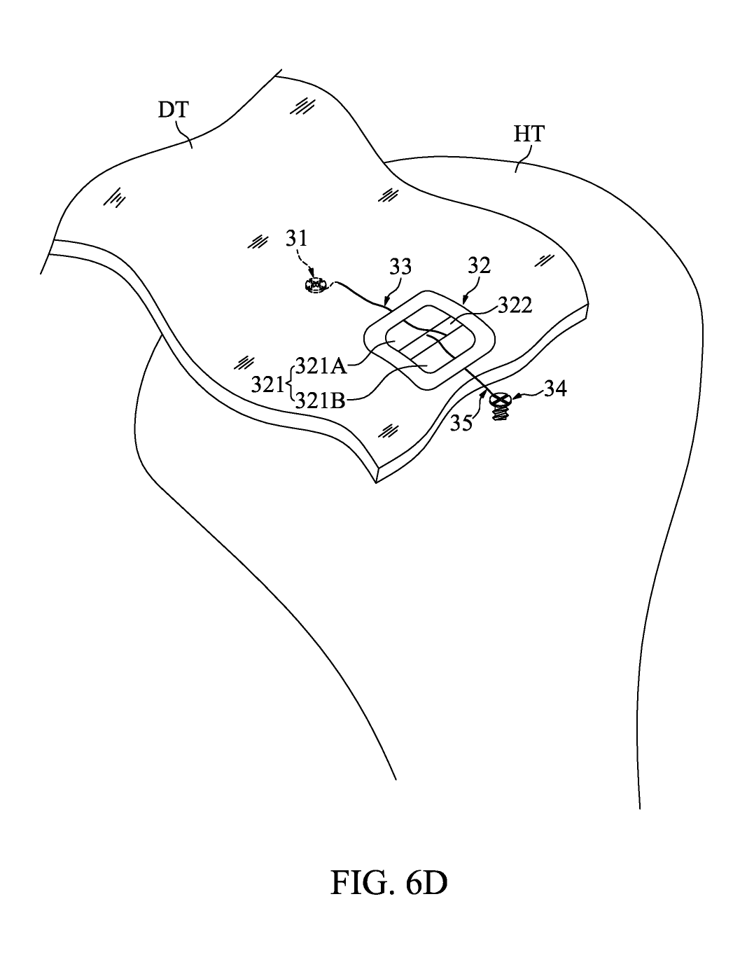

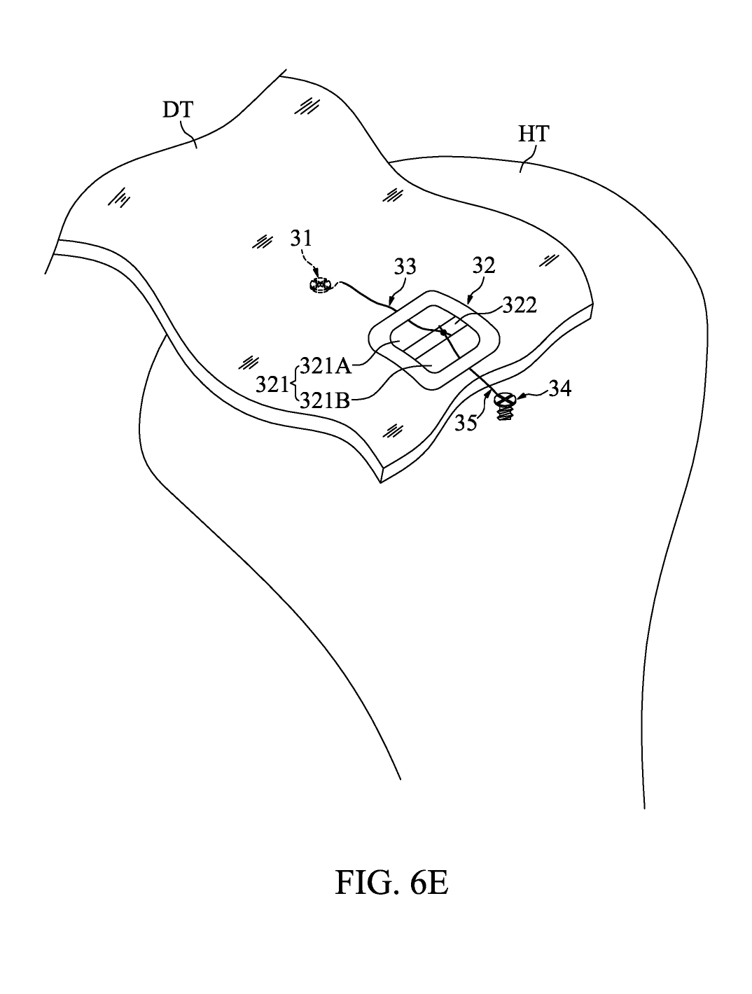

In FIG. 6A, a suture anchor 31 is fixed on a hard tissue HT, and at least one first suture 33 is secured to the suture anchor 31 and penetrates a detached tissue DT.

Referring to FIG. 6B, a fastener 34 is fixed on the hard tissue HT, and at least one second suture 35 is secured to the fastener 34. In some embodiments, the fastener 34 can be a suture anchor or a pushlock.

Referring to FIG. 6C, at least one button 32 is configured to press the detached tissue DT on the hard tissue HT. The button 32 is disposed between the suture anchor 31 and the fastener 34. The button 32 includes a hollow portion 321 and a bisecting rod 322 disposed within the hollow portion 321 for dividing the hollow portion 321 into a first hollow portion 321A and a second hollow portion 321B.

Referring to FIG. 6D, the first suture 33 penetrates the first hollow portion 321A of the button 32 and the second suture 35 penetrates the second hollow portion 321B of the button 32.

Referring to FIG. 6E, the second suture 35 and the first suture 33 are knotted on the bisecting rod 322 of the button 32, thereby generating a pressure on the button 32 to uniformly press the detached tissue DT on the hard tissue HT.

Moreover, the scope of the present application is not intended to be limited to the particular embodiments of the process, machine, manufacture, and composition of matter, means, methods and steps described in the specification. As those skilled in the art will readily appreciate form the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure.

Accordingly, the appended claims are intended to include within their scope such processes, machines, manufacture, and compositions of matter, means, methods or steps. In addition, each claim constitutes a separate embodiment, and the combination of various claims and embodiments are within the scope of the invention.

What is claimed is:

1. An apparatus for reattaching a detached tissue to a hard tissue, comprising:
a suture anchor fixed on a hard tissue;
at least one button configured to press a detached tissue on the hard tissue, wherein the button includes a hollow portion and a bisecting rod disposed within the hollow portion for dividing the hollow portion into a first hollow portion and a second hollow portion;
at least one first suture secured to the suture anchor and penetrating the detached tissue and the first hollow portion of the button;
a fastener fixed on the hard tissue; and
at least one second suture secured to the fastener and penetrating the second hollow portion of the button, wherein the second suture and the first suture are knotted on the bisecting rod of the button, thereby generating a pressure on the button to uniformly press the detached tissue on the hard tissue.

2. The apparatus of claim 1, wherein the button includes a sawtooth structure contacted with the detached tissue.

3. The apparatus of claim 1, wherein the fastener is a suture anchor or a pushlock.

4. A method for reattaching a detached tissue to a hard tissue, comprising:
fixing a suture anchor on a hard tissue and securing at least one first suture to the suture anchor, wherein the first suture penetrates a detached tissue;
fixing a fastener on the hard tissue and securing at least one second suture to the fastener;
configuring at least one button to press the detached tissue on the hard tissue, wherein the button includes a hollow portion and a bisecting rod disposed within the hollow portion for dividing the hollow portion into a first hollow portion and a second hollow portion;
enabling the first suture to penetrate the first hollow portion of the button and enabling the second suture to penetrate the second hollow portion of the button; and
knotting the second suture and the first suture on the bisecting rod of the button, thereby generating a pressure on the button to uniformly press the detached tissue on the hard tissue.

5. The method of claim 4, wherein the button includes a sawtooth structure contacted with the detached tissue.
